(19) United States
(12) Patent Application Publication
(10) Pub. No.: US 2021/0153623 A1
(43) Pub. Date: May 27, 2021

(54) SKIN CARE BEAUTY PACK FOR HAND OR FOOT AND MANUFACTURING METHOD THEREOF

(71) Applicant: EM Corporation, Hwaseong-si (KR)

(72) Inventor: Han-Wook PARK, Seoul (KR)

(21) Appl. No.: 17/101,020

(22) Filed: Nov. 23, 2020

(30) Foreign Application Priority Data
Nov. 22, 2019 (KR) 10-2019-0151304

(51) Int. Cl. A45D 34/04; B32B 5/02; B32B 27/12; B32B 7/14; A41D 19/00; A41B 11/00; A61K 8/02; A61Q 19/00

(52) U.S. Cl. CPC A45D 34/04; B32B 5/022; B32B 27/12; B32B 7/14; A41D 19/0006; A45D 2200/1027; A41D 19/0065; A41B 11/00; A61K 8/0212; A61Q 19/007; A45D 2200/1036; A41D 19/0044

(57) ABSTRACT

The present invention relates to a skin care beauty pack for a hand and a foot formed of a glove- or Korean-sock-shaped pack body that includes outer upper and lower films and inner upper and lower nonwoven fabrics and in which cosmetic essence is impregnated between the upper and lower nonwoven fabrics and heat-pressed, wherein the pack body 8 is completely sealed by the neck portion 4 and the edge seam 6 such that the cosmetic essence may not be leaked, and since the pattern bonding portion 10 for respectively bonding the outer upper film 2a and the inner upper nonwoven fabric 12a, and the outer lower film 2b and the inner lower nonwoven fabric 12b, is formed near the neck portion 4, the skin beauty pack that may reliably and easily opened at the neck portion 4, and the manufacturing method of the skin care beauty pack in an economical manner that does not cause an increase in cost by adding only equipment required for the formation of the pattern bonding portion 10, is provided.

### CROSS-REFERENCE TO RELATED APPLICATION

This application claims priority to and the benefit of Korean Patent Application No. 10-2019-0151304 filed in the Korean Intellectual Property Office on Nov. 22, 2019, the entire contents of which are incorporated herein by reference.

### BACKGROUND OF THE INVENTION

### (a) Field of the Invention

The present invention relates to a skin care beauty pack for a hand and a foot that is used for skin care of hands or feet, and a manufacturing method thereof.

### (b) Description of the Related Art

Beauty packs that promote facial skin care by covering a face with a nonwoven fabric impregnated with beauty essences to moisturize and supply nutrients to a skin are widely spreading, and an application range of such a pack is extended to skin care of a hand and a foot through a hand pack and a foot pack.

Several types of hand pack or foot pack are known in the following patent documents.

In the hand pack or foot pack disclosed in Patent Document 1, a tightening member having an adhesive band is attached to one side of a wrist portion or an ankle portion, and thus, when it is used, it prevents leakage of cosmetic essence by tightening an opened wrist or ankle portion.

In addition, Patent Document 2 discloses a feature in which a hand pack or foot pack is provided with a Velcro tape attached so as to prevent leakage of cosmetic essence through an opened portion during use, and a feature in which a beauty pack for an arm or leg portion is further provided so as to promote skin care beauty of the arm or leg portion.

Patent Document 3 discloses a feature in which a pair of hand packs or foot packs are arranged in a symmetrical fashion, and a feature in which a cut-out portion made of a sealing treatment portion including a dotted line for cutting is provided as a boundary between the pair of hand packs or foot packs such that it may be separated at the dotted line by cutting, and the following Patent Documents 4 to 7 similarly disclose examples in which a pair thereof are symmetrically arranged.

In addition, Patent Document 8 discloses an example in which a heat-shrinkable band is attached to a portion corresponding to a wrist or ankle such that an internal cosmetic essence is not leaked by heat shrinking during use.

Structural features of the skin care beauty packs for hands and feet known through the patent documents are classified into the feature that the beauty essence is prevented from leaking through the wrist or ankle portion when worn and the feature in which a pair of hand packs or foot packs are formed in a symmetrical fashion and they are provided with a dotted line for cutting on a boundary therebetween such that they are separated along the dotted line when used, and then the separated packs are worn on both hands or both feet.

However, as described above, in the skin care beauty pack for hands and feet with the dotted line for cutting, there is a drawback in which the cosmetic essence impregnated in the internal nonwoven fabric leaks through at the dotted line.

For this reason, in the skin care beauty packs with dotted lines for cutting, a gap between the impregnated position of the cosmetic essence and the dotted line for cutting is disposed as far away as possible, so that an attempt is made to delay starting of leakage, but this is not the best solution.

In addition to the structure that provides dotted lines for cutting to the skin care beauty pack, in the commercially available hand pack or foot pack, the edge excluding the wrist or ankle portion is strongly heat-compressed such that the outer film and the inner nonwoven fabric are integrally bonded, and the wrist or ankle portion is weakly heat-compressed such that only the inner nonwoven fabric is bonded.

As such, since the microstructure is entangled in the portions of the nonwoven fabrics bonded to each other, when the internal cosmetic essence permeates thereinto, it may leak through a gap of the microstructure due to the capillary phenomenon.

The leakage problem of the cosmetic essence as described above may be completely prevented by strongly thermally compressing and sealing between the outer film and the inner nonwoven fabric.

However, since the films of the strongly heat-compressed portions are also bonded to each other at the edges thereof, when a user pulls and opens it by hand, as shown in a photograph in FIG. 1, the films are stretched and not opened, so tools such as scissors are required, causing an inconvenience.

### PRIOR ART DOCUMENT

### Patent Document

(Patent Document 1) Korean Utility Model No. 20-2010-0006356
(Patent Document 2) Korean Registered Utility Model No. 20-0478460
(Patent Document 3) Korean Registered Utility Model No. 20-0458722
(Patent Document 4) Korean Registered Utility Model No. 20-0466462
(Patent Document 5) Korean Registered Utility Model No. 20-0464826
(Patent Document 6) Korean Registered Utility Model No. 20-0458722
(Patent Document 7) Korean Registered Utility Model No. 20-0464833
(Patent Document 8) Korean Registered Utility Model No. 20-0468309

The above information disclosed in this Background section is only for enhancement of understanding of the background of the invention, and therefore it may contain information that does not form the prior art that is already known in this country to a person of ordinary skill in the art.

### SUMMARY OF THE INVENTION

An aspect of the present invention provides a skin care beauty pack for a hand and a foot that completely eliminates a problem of leakage of cosmetic essences impregnated into an inner nonwoven fabric of a hand pack or foot pack, and that may be easily and reliably opened even by grabbing and pulling any portion of a neck side thereof when opening.

In addition, another aspect of the present invention provides a manufacturing method of a skin care beauty pack for a hand and a foot that may completely seal the skin care beauty pack so that an inner cosmetic essence does not leak, and that may reliably and easily open the skin care beauty pack.

An embodiment of the present invention provides a skin care beauty pack for a hand and a foot that includes: a pack body including a neck portion and a seam portion that are completely sealed by heat pressurization in a shape of gloves or Korean socks; and a pattern bonding portion that is formed at a periphery of the neck portion and integrally bonds an upper film and an upper nonwoven fabric, and a lower film and a lower nonwoven fabric.

In the skin care beauty pack for the hand and the foot, the neck portion may be bonded and sealed at cut surfaces of the upper and lower films.

Another embodiment of the present invention provides a manufacturing method of a skin care beauty pack for a hand and a foot, including: a process of overlapping a film and a nonwoven fabric, and repeatedly forming a pattern bonding portion in which the film and the nonwoven fabric are integrally bonded at predetermined positions by heat pressing at a film side; a process of injecting cosmetic essence while overlapping the film and the nonwoven fabric in which the pattern bonding portion is formed in pairs and overlapping the nonwoven fabrics to face each other at the inside; and a process of forming a pack body in which the edge seam portion and the neck portion are completely sealed by heat-pressing while controlling the pattern bonding portion to be positioned around the neck portion of a glove or Korean-sock shape.

According to the skin care beauty pack for the hand and the foot of the embodiment of the present invention described above, since an entire edge including a wrist or ankle portion is completely sealed by a seam portion that integrally bonds a film and a nonwoven fabric, cosmetic essences injected into an inner space thereof do not leak, and even if it is left for a long time, it remains completely sealed, thus it is possible to fundamentally solve the problem of product defects caused by leakage of cosmetic essences in the related art.

In addition, according to the skin care beauty pack for the hand and the foot of the embodiment of the present invention, since a film and a nonwoven fabric are integrally bonded by a pattern bonding portion formed near a neck portion of a glove or Korean-sock shape, even if a user grabs and pulls upper and lower films thereof, the nonwoven fabric is pulled together from the inside, so that it may be reliably and easily opened, thus it has the advantage of being convenient to use.

Further, according to the manufacturing method of the skin care beauty pack for the hand and the foot of the embodiment of the present invention, since only a process of heat pressing a pattern bonding portion between a film and a nonwoven fabric is added, the skin care beauty pack may be implemented with a low equipment cost without causing an increase in product cost.

### BRIEF DESCRIPTION OF THE DRAWINGS

FIG. 1 is a photograph of a form in which bonded films are pulled and stretched when the fully sealed pack body is opened.

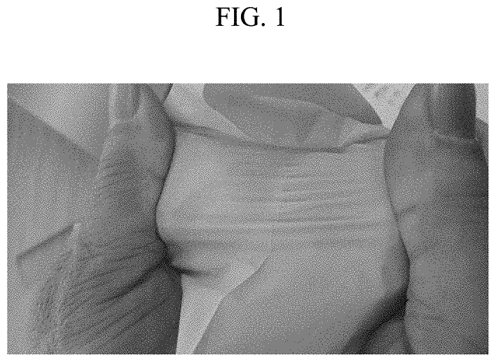

FIG. 2 illustrates a perspective view of a skin care beauty pack for a hand and a foot according to an embodiment of the present invention.

FIG. 3 illustrates a cross-sectional view taken along line III-III of FIG. 2.

FIG. 4 illustrates a cross-sectional view taken along line IV-IV of FIG. 2.

FIG. 5 illustrates a perspective view of a skin care beauty pack for a hand and a foot according to an embodiment of the present invention, and is a drawing corresponding to FIG. 2.

FIG. 6 is a photograph of an open state of a hand pack to which the present invention is applied.

FIG. 7 is a photograph of an open state of a foot pack to which the present invention is applied.

FIG. 8 is a drawing for explaining a manufacturing method of the present invention.

### DETAILED DESCRIPTION OF THE EMBODIMENTS

As those skilled in the art would realize, the described embodiments may be modified in various different ways, all without departing from the spirit or scope of the present invention.

In order to clearly describe the present invention, parts that are irrelevant to the description are omitted, and identical or similar constituent elements throughout the specification are denoted by the same reference numerals.

In addition, unless explicitly described to a first contrary, the word “comprise” and variations such as “comprises” or “comprising” will be understood to imply the inclusion of stated elements but not the exclusion of any other elements.

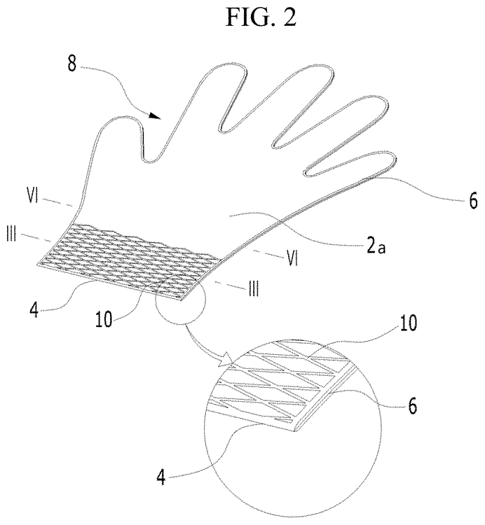

A skin care beauty pack for a hand and a foot according to an embodiment of the present invention will be described in detail with reference to FIG. 2 to FIG. 8.

FIG. 2 illustrates a perspective view of a skin care beauty pack for a hand and a foot according to an embodiment of the present invention, and particularly, it illustrates a glove-shaped pack body 8 in which the outside thereof is covered with upper and lower films 2a and 2b, and a neck portion 4 and an edge seam 6 are cut simultaneously while hot pressing to be completely sealed.

It is preferable to form the neck portion 4 short without a width by performing bonding of the neck portion 4 at the cut surface, and the seam 6 is not necessarily required to be, but it is preferable to firmly bond the seam 6 so that a pressed width appears, so that it does not unintentionally burst at an edge thereof.

When a tip angle of a heating knife performing heat pressing is in a range of 60 to 75 degrees with respect to a vertical surface, in bonding at the cutting surface, the neck portion 4 may be weakly bonded with a narrow width, and thus it may be opened with even the weak strength of the elderly.

The illustrated pack body 8 is provided with a pattern bonding portion 10 near the neck portion 4.

The pattern bonding portion 10 may be formed by repeatedly arranging bonding portions of a predetermined shape. That is, the pattern bonding portion 10 may be formed with a constant motif.

In the present embodiment, the pattern bonding portion 10 has a shape in which a number of rhombuses formed by lines intersecting in a diagonal direction are arranged in four directions, but is not limited thereto, and it may have a shape in which a number of circles, quadrangles, hexagons, etc. are arranged in succession in four or two directions, or it may have a pattern in which vertical or horizontal straight lines are arranged continuously at regular intervals.

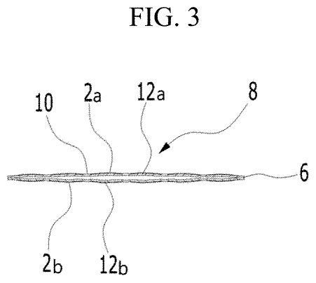

As shown in FIG. 3, the pattern portion 10 is one in which the outer upper film 2a and an inner upper nonwoven fabric 12a, and the outer lower film 2b and an inner lower nonwoven fabric 12b are respectively heat-pressed to be integrally bonded.

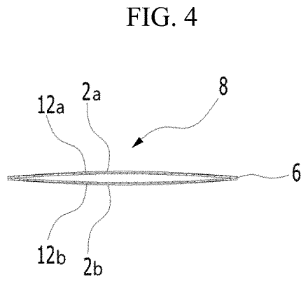

In a portion of the pack body 8 excluding the pattern bonding portion 10, as illustrated in detail in FIG. 4, the upper film 2a and the upper nonwoven fabric 12a, and the lower film 2b and the lower nonwoven fabric 12b, are overlapped while being separated from each other in the same manner as a typical skin care beauty pack, and the upper and lower nonwoven fabrics 12a and 12b are separated from each other to form a space therebetween.

In the skin care beauty pack for the hand and the foot of the present invention described above, since the pack body 8 is completely sealed by the neck portion 4 and the edge seam 6, the cosmetic essence injected therein does not leak to the outside.

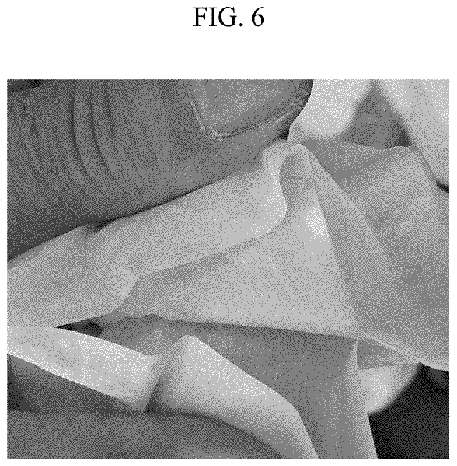

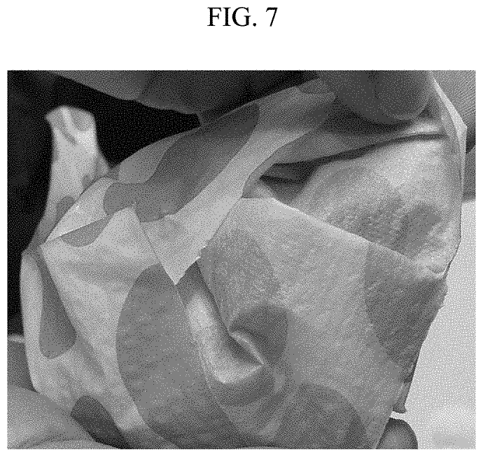

In use, when the neck portion 4 or a periphery thereof is held and pulled by hand, since the outer upper and lower films 2a and 2b and the inner upper and lower nonwoven fabrics 12a and 12b are pulled together and opened, as shown in FIG. 6 and FIG. 7, the outer upper and lower films 2a and 2b are not stretched and are opened cleanly and reliably together with the inner upper and lower nonwoven fabrics 12a and 12b.

Therefore, the skin care beauty pack for the hand and the foot according to the present invention completely solves the problem of leakage of the cosmetic essence in the conventional skin care beauty pack, and it is convenient to use as the completely sealed neck portion may be opened cleanly and reliably without an effort.

In addition, when the pack body 10 is worn, since the upper and lower nonwoven fabrics 12a and 12b separated from the upper and lower films 2a and 2b are in close contact with the skin of the hand or foot, the delivery to the skin of the cosmetic essence impregnated therein is maintained at an excellent level, and it is also comfortable to wear, so it is not unnatural.

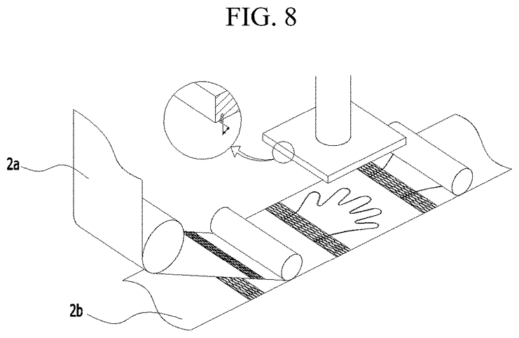

Hereinafter, a manufacturing process of the skin care beauty pack for the hand and the foot will be described with reference to FIG. 8.

A film is overlapped on a nonwoven fabric and thermally pressurized at a predetermined position to form a continuous pattern bonding portion 10 to obtain a fabric material C.

The fabric material C is employed as the upper film 2a and the upper nonwoven fabric 12a, or the lower film 12b and the lower nonwoven fabric 12b, which are integrally bonded by the pattern bonding portion 10.

The lower nonwoven fabric 12b is unfolded upward, and the cosmetic essence is injected thereon, and then the upper nonwoven fabric 12a is overlapped so as to face the lower nonwoven fabric 12b.

In this case, by heat-pressing the upper and lower pattern bonding portions 10 after the upper and lower pattern bonding portions 10 are adjusted so that their positions are matched and are adjusted to be positioned around the neck portion 4 in a shape of the hand and foot pack of a glove or Korean-sock shape, the pack body 8 in which the edge seam 6 and the neck portion 4 are completely sealed is formed and manufactured.

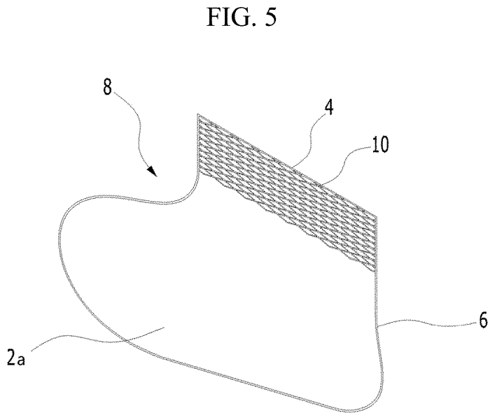

In the present embodiment, the pack body 8 described above is exemplified as a hand pack, but as shown in FIG. 5, the pack body 8 may be implemented as a Korean-sock-shaped foot pack. FIG. 5 illustrates a foot pack as a perspective view of a skin care beauty pack for a hand and a foot according to another embodiment of the present invention, and is a drawing corresponding to FIG. 2.

Even in the case of being implemented as the foot pack, when being opened for use, the pattern bonding portion 10 is positioned near the neck portion 4 so that the outer upper and lower films 2a and 2b and the inner upper and lower nonwoven fabrics 12a and 12b are pulled together, and other portions are formed in the same structure in which the upper and lower films 2a and 2b and the upper and lower nonwoven fabrics 12a and 12b are separated and overlapped.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A skin care beauty pack for a hand and a foot in which a film is applied to the outside of two nonwoven fabrics impregnated with cosmetic essence and heat-pressurized and then cut into a glove or Korean-sock shape, comprising:
a pack body 8 that is completely sealed by a glove or Korean-sock shaped neck portion 4 and an edge seam 6 and in which a pattern bonding portion 10 that integrally bonds an outer upper film 2a and an inner upper nonwoven fabric 12a, and an outer lower film 2b and an inner lower nonwoven 12b, is formed around the neck portion 4.

2. The skin care beauty pack for the hand and the foot of claim 1,
wherein the neck portion 4 is bonded and sealed at cut surfaces of the upper and lower films 2a and 2b.

3. A manufacturing method of a skin care beauty pack for a hand and a foot, comprising:
a process of overlapping a film and a nonwoven fabric, and repeatedly forming a pattern bonding portion in which the film and the nonwoven fabric are integrally bonded at predetermined positions by heat pressing at a film side;
a process of injecting cosmetic essence while overlapping the film and the nonwoven fabric in which the pattern bonding portion is formed in pairs and overlapping the nonwoven fabrics to face each other in the inside; and
a process of forming a pack body in which the edge seam portion and the neck portion are completely sealed by heat-pressing while controlling the pattern bonding portion to be positioned around the neck portion of a glove or Korean-sock shape.
